(19) United States
(12) Patent Application Publication
(10) Pub. No.: US 2020/0170571 A1
(43) Pub. Date: Jun. 4, 2020

(54) ANALYSIS DEVICE FOR ANALYZING EXPIRATION AIR

(71) Applicant: B. BRAUN MELSUNGEN AG, MELSUNGEN (DE)

(72) Inventor: GABRIELE SPRAVE-BAUMBACH, Reutlingen (DE)

(21) Appl. No.: 16/616,057

(22) PCT Filed: May 24, 2018
(86) PCT No.: PCT/EP2018/063720

§ 371 Date: Nov. 22, 2019

(30) Foreign Application Priority Data
May 24, 2017 (DE) 10 2017 111 459.9

(51) Int. Cl. A61B 5/00; A61B 5/08; G01N 27/62; G01N 33/497

(52) U.S. Cl. CPC A61B 5/4821; A61B 5/082; A61B 2560/0223; G01N 33/497; G01N 27/622

(57) ABSTRACT

An analysis device for analyzing expiration air of a patient, preferably for monitoring a patient under anesthesia during a medical intervention, is configured for determining, in the expiration air, a portion of an analyte contained in the expiration air. The analysis device preferably has a multi-capillary column for separating the expiration air to be analyzed and an ion mobility spectrometer in which gas components of the expiration air are ionized and accelerated toward a detection device. The analysis device can output signal excursions which are created by the ionized gas components of the expiration air which hit the detection device. A portion of the analyte which is to be determined and is contained in the expiration air to be analyzed is determined by a calibration of the signal excursion of the analyte to a signal excursion which is caused by the air moisture of expiration air.

### CROSS REFERENCE TO RELATED APPLICATIONS

This application is the United States national phase entry of International Application No. PCT/EP2018/063720, filed May 24, 2018, which claims the benefit of priority of German Application No. 10 2017 111 459.9, filed May 24, 2017. The contents of International Application No. PCT/EP2018/063720 and German Application No. 10 2017 111 459.9 are incorporated by reference herein in their entireties.

### FIELD

The invention relates to an analysis device for analyzing expiration air of a patient, preferably for monitoring a patient under anesthesia during a medical intervention, the analysis device being configured for determining, in the expiration air, a portion of an analyte contained in the expiration air of the patient, having: preferably a multi-capillary column for separating the expiration air to be analyzed; and an ion mobility spectrometer in which gas components of the expiration air are ionized and accelerated toward a detection device; the analysis device outputting signal excursions which are created by the ionized gas components of the expiration air which hit the detection device.

### BACKGROUND

From the state of the art, ion mobility spectrometers are known which serve for detecting chemical substances, warfare agents, explosives, drugs etc. Also, it is known e.g. from DE 20 2013 105 685 U1 to use such ion mobility spectrometers in the field of medicine, for example for monitoring anesthesia during medical interventions. In so doing, an anesthetic, such as e.g. propofol, is continuously analyzed in a patient's breathing air.

Moreover, from the state of the art, analysis devices are known that have an ion mobility spectrometer in combination with an upstream multi-capillary column. A multi-capillary column is a gas-chromatographic column consisting of a plurality of bundled individual capillaries that retain different analytes for a differently long time. In other words, the gas components of the expiration air take differently long to pass through the multi-capillary column so that an expiration air sample can be pre-separated by means of the multi-capillary column (1^{st} separation). A passage time through the multi-capillary column is referred to as retention time.

After the pre-separation in the multi-capillary column the gas components reach the ion mobility spectrometer, namely initially an ionizing chamber portion of the ion mobility spectrometer in which the gas components of the expiration air are ionized. This is carried out with the aid of an ion source, for example by means of radioactive nickel. After ionization, the ions pass a barrier grid and are accelerated in a drift chamber portion of the ion mobility spectrometer against the flow direction of a drift gas toward a detection device. Ions of different mass and, resp., structure reach different drift velocities here, are thus separated from each other (2^{nd} separation) and successively hit the detection device. A passage time through the drift chamber section is referred to as drift time. The acceleration of the ions in the ion mobility spectrometer takes place by means of an electric field. The analysis device outputs signal excursions which are created by the ionized gas components of the expiration air that hit the detection device.

In the state of the art, the attempts to provide analysis devices of this type for use with the patient during a medical intervention have not been successful in appropriately handling the high relative moisture of expiration air. So far, the high moisture of expiration air has always been considered to be detrimental to the measuring results. The state-of-the-art documents merely indicate solutions as to how the influences due to moisture can be reduced/avoided and useful measuring results can be achieved despite the high moisture of expiration air. Furthermore, the state of the art shows the drawback that the analysis device can be used, after being turned on, as late as after a period of several days for quantitative measurements for analyzing expiration air, as during said period of time the signal excursions output are continuously increasing and only after that will even out to a constant value.

### SUMMARY

Against this background, it is the object of the present invention to provide an analysis device of compact design and optimized for application for analyzing expiration air of a patient, preferably for monitoring a patient under anesthesia during a medical intervention by means of which reliable exact and continuous measurements for repeat accuracy over a quite long period can be carried out immediately after turning on the analysis device.

The invention relates to an analysis device for analyzing expiration air of a patient, preferably for monitoring a patient under anesthesia during a medical intervention, the analysis device being configured for determining, in the expiration air, a portion of an analyte contained in the expiration air of the patient, comprising: preferably a multi-capillary column for pre-separating the expiration air to be analyzed; and an ion mobility spectrometer in which gas components of the expiration air are ionized and accelerated toward a detection device; the analysis device outputting signal excursions which are created by the ionized gas components of the expiration air which hit the detection device. A portion of the analyte which is to be determined and is contained in the expiration air to be analyzed is determined by a calibration of the signal excursion of the analyte to a signal excursion which is caused by the air moisture of expiration air.

It is of advantage when a maximum signal excursion of the analyte is put in relation to a maximum signal excursion of the signal excursion caused by the air moisture of expiration air and the portion of the analyte in the expiration air is determined on the assumption of a known and constant relative air moisture of expiration air of the patient, especially a relative air moisture of 95% or a relative air moisture established in advance for a specific patient.

Advantageously, the analysis device is configured to carry out measurement by proportion of the analyte in the expiration air with continuously increasing absolute signal excursions and thus directly after turning on the analysis device by calibrating the signal excursion of the analyte to the signal excursion caused by the air moisture of the expiration air.

In other words, according to the present invention, initially an absolute quantitative maximum value of a signal excursion which is created by the air moisture of expiration air is established.

Due to the high moisture of expiration air, said value can be established relatively easily as merely the maximum signal excursion output by the analysis device must be looked for. It is finally assumed that said absolute value corresponds to a known and constant moisture portion of expiration air of the patient. This moisture portion can always be set to a fixed value, for example 95%, or can be established for a specific patient prior to the respective medical intervention.

When the portion of a specific analyte in the expiration air is to be determined, only an absolute quantitative maximum value of a signal excursion of the analyte must be established. Then the portion of the analyte in the expiration air can be calculated by a rule-of-three relationship. Said described back-calculation to the portion of the analyte is referred to as calibration in the present invention.

Hence, the core of the invention resides in the fact that the known and constant moisture portion of expiration air of the patient is utilized for determining the portion of a specific analyte. Furthermore, it is the core of the invention that for determining the portion of the analyte merely the ratio between the maximum signal excursion of the analyte and the maximum signal excursion due to moisture has to be determined. Thus, the analysis device according to the invention can operate even at continuously increasing absolute values, i.e. immediately after turning on the analysis device.

Of preference, the analyte to be determined is an anesthetic, preferably propofol. When a portion of an anesthetic is determined in the expiration air of a patient, the anesthetic intensity can be concluded and thus the patient under anesthesia during a medical intervention can be monitored.

It is of advantage when the analysis device is configured to establish the portion of the anesthetic in the expiration air at predetermined short time intervals, especially at least every five minutes, preferably every two minutes, further preferred every minute, and to indicate the measuring values obtained on a display.

An advantageous example embodiment is characterized in that the gas components of the expiration air take differently long for passing through the multi-capillary column and a passage time through the multi-capillary column is referred to as retention time; the ion mobility spectrometer has an ionization chamber portion in which the gas components of the expiration air are ionized and a drift chamber portion in which the ionized gas components are accelerated toward the detection device and a passage time through the drift chamber portion is referred to as drift time; and the analysis device outputs the signal excursions in a chromatogram as a function of the retention time and the drift time.

Of preference, the signal excursion caused by the air moisture of expiration air is provided substantially independently of the retention time after a particular drift time in the chromatogram and represents especially a maximum signal excursion in the chromatogram.

Advantageously, the signal excursion caused by the air moisture of the expiration air is created by reactions ions, especially H⁺(H₂O)ⁿ ions or O₂⁻(H₂O)ⁿ ions, formed during ionization of the expiration air and hitting the detection device, which excel by a characteristic signal excursion in the chromatogram.

Of preference, the analysis device has a data base in which two values each for the drift time and the retention time are stored for different analytes, wherein the four values stored in the data base define a range in the chromatogram in which the signal excursion for a particular analyte is provided, wherein a drift time axis is preferably standardized to the signal excursion caused by the air moisture of expiration air.

### BRIEF DESCRIPTION OF THE DRAWING FIGURES

The invention shall be further explained hereinafter by way of figures, wherein:

FIG. 1 shows a schematic view of an analysis device according to the invention;

FIG. 2 shows a three-dimensional view of a chromatogram in which signal excursions are shown as a function of a drift time and a retention time; FIG. 3 shows a two-dimensional view of a chromatogram in which signal excursions are shown

as a function of the drift time and the retention time;

FIG. 4 shows a display of the analysis device according to the invention; and

FIG. 5 shows a flow diagram of steps to be carried out until the analysis device according to the invention is ready for measuring.

### DETAILED DESCRIPTION

The figures a merely schematic and serve exclusively for the comprehension of the invention. Like elements are provided with like reference numerals.

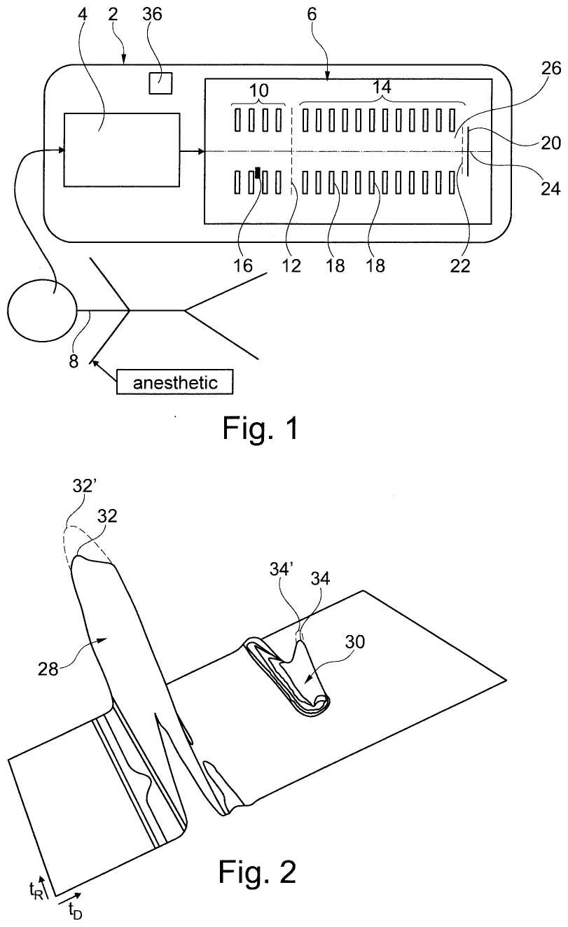

FIG. 1 illustrates a schematic view of an analysis device 2 according to the invention. The analysis device 2 includes a multi-capillary column 4 and an ion mobility spectrometer 6. The multi-capillary column 4 consists of a plurality of bundled individual capillaries (not shown). Different gas components of expiration air take differently long for passing through the multi-capillary column 4. Expiration air which is expired by a patient 8 and is supplied to the multi-capillary column 4, as shown in FIG. 1, thus is divided into individual gas components with the aid of the multi-capillary column. The time required by a gas component for passing through the multi-capillary column 4 is referred to as retention time t_{R}.

After a first separation by means of the multi-capillary column 4 the expiration air and, resp., the gas components thereof is/are supplied to the ion mobility spectrometer 6. The ion mobility spectrometer 6 comprises an ion chamber portion 10 and a drift chamber portion 14 adjacent thereto and separated from the ionization chamber portion 10 by a Bradbury-Nielsen grid 12. In the ionization chamber portion 10 the gas components of the expiration air are ionized by means of an ionization source 16 (for example radioactive nickel). The Bradbury-Nielsen grid 12 controls penetration of the ions generated in the ionization chamber portion 10 into the drift chamber portion 14. Via an electric field generated by means of high-voltage rings 18 the ions are accelerated toward a Faraday plate 20 which serves for detecting the ions. Directly ahead of the Faraday plate 20, an aperture grid 22 is provided as a shielding grid for capacitive uncoupling of the ions. On the side of the drift chamber portion 14 including the Faraday plate 20, an inlet opening 24 for a drift gas is provided which flows through an interior 26 opposite to a drift direction of the ions and prevents uncharged molecules or particles from entering into the drift chamber portion 14.

Ions of different mass and, resp., structure reach different drift velocities in the drift chamber portion 14, are thus separated from one another (second separation) and successively hit the

Faraday plate 20. A passage time of the ions through the drift chamber portion 14 is referred to as drift time t_{D}.

The analysis device 2 is configured to determine, in the expiration air, a portion of an analyte contained in the expiration air. In the present invention, the analyte to be determined preferably is an anesthetic, preferably propofol, which was administered intravenously to the patient 8 and which the latter exhales under anesthesia via the expiration air.

The accuracy with which the portion of the analyte contained in the expiration air is determined according to the invention is explained with reference to FIG. 2. FIG. 2 illustrates a chromatogram which exemplifies two signal excursions that are created by ionized gas components hitting the Faraday plate 20 and are output by the analysis device 2 as a function of the retention time t_{R} and the drift time t_{D}.

FIG. 2 illustrates a first signal excursion 28 and a second signal excursion 30. The first signal excursion 28 is caused by the air moisture of the expiration air, especially by reaction ions, especially H⁺(H₂O)ⁿ ions or O₂⁻(H₂O)ⁿ ions, formed when the expiration air is ionized and hitting the Faraday plate 20. Said first signal excursion 28 is present substantially independently of the retention time after a particular drift time in the chromatogram and constitutes, due to the high relative moisture of expiration air of more than 95%, in an analysis of expiration air always a maximum characteristic signal excursion in the chromatogram.

The second signal excursion 30 is caused by an analyte (for example an anesthetic, preferably propofol) the portion of which in the expiration air has to be determined.

According to the invention, initially a first maximum (absolute quantitative value) 32 of the first signal excursion 28 is determined. Subsequently, a second maximum (absolute quantitative value) 34 of the second signal excursion 30 is determined. To obtain the portion of the analyte in the expiration air, for example the ratio between the second maximum 34 and the first maximum 32 is formed and is multiplied with the known and constant air moisture portion of the expiration air of the patient 8. In other words, according to the invention calibration of the second maximum 34 of the second signal excursion 30 to the first maximum 32 of the first signal excursion 30 is carried out.

In FIG. 2, broken lines indicate another first maximum 32′ of the first signal excursion 28 and a second maximum 34′ of the second signal excursion 30. The first maximum 32′ and the second maximum 34′ would be obtained, if the same expiration air sample was supplied once again to the analysis device 2 to a later point in time. In other words, basically with an increasing period of time after turning on the analysis device 2 increasing maximums 32′, 34′ of the signal excursions 28, 30 are obtained. The present invention allows to appropriately treat continuously increasing absolute signal excursions as, according to the invention, in each case only the ratio between the second maximum 34, 34′ and the first maximum 32, 32′ has to be formed.

FIG. 3 illustrates a two-dimensional view of the chromatogram obtained in which different signal excursions obtained, for example again the signal excursions 28 and 30 shown in FIG. 2, are shown. As explained already before, the signal excursion 28 represents a signal excursion characteristic of the moisture and can be found in a simple way as it is provided after a particular drift time t_{D} independently of the retention time t_{R}.

The analysis device 2 includes a data base 36 in which two values each for the drift time t_{D} and the retention time t_{R} are stored for different analytes to be determined. In the chromatogram said four values define a rectangular area 38 in which the signal excursion for a particular analyte is provided. Of preference, the values for the drift time t_{D} are standardized to the first characteristic signal excursion 28.

When the second maximum 34 of the second signal excursion 30 is to be determined, the rectangular area 38 is defined in the chromatogram by the values obtained in the data base 36 and only the maximum/the maximum absolute value has to be determined in the defined rectangular area 38.

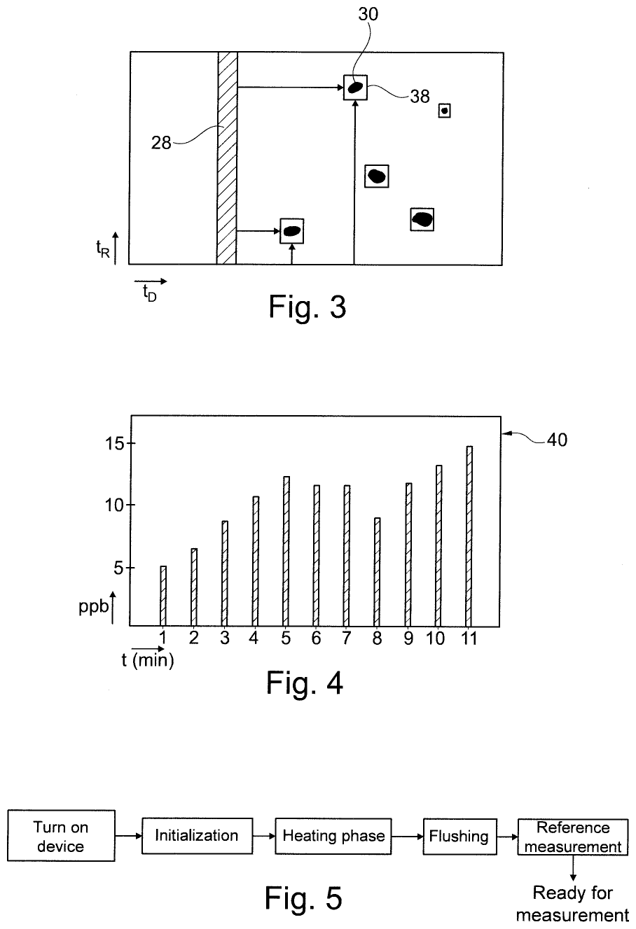

As shown in FIG. 4, the analysis device 2 measures/calculates/establishes, for example, every minute a portion ppb (parts per billion) of an analyte, preferably of an anesthetic, in the expiration air of a patient and outputs the portion obtained in each case on a display. Thus, a physician is allowed to continuously analyze the portion of the anesthetic (propofol) in the expiration air and to monitor the patient under anesthesia during a medical intervention. For example, the physician can repeatedly administer the anesthetic intravenously to the patient 8, when he/she notices after 6 minutes or 7 minutes as shown in FIG. 4 that the portion of the anesthetic in the expiration air subsides.

FIG. 5 illustrates a flow diagram of steps to be carried out until the analysis device 2 according to the invention is ready for measurement. Turning on the device is followed by initialization, a heating phase, flushing and a reference measurement. Said steps take less than 30 minutes. Where it has been mentioned in the foregoing that measurement of a portion of the analyte in the expiration air can be carried out immediately after turning on the analysis device 2, this shall mean that measurement of a portion can be carried out after 30 minutes at the latest.

1. An analysis device adapted for analyzing expiration air of a patient, the analysis device being configured to determine, in the expiration air, a portion of an analyte in the expiration air, the analysis device comprising:
at least one multi-capillary column adapted for pre-separating the expiration air; and
an ion mobility spectrometer in which gas components of the expiration air are ionized and accelerated towards a detection device,
the analysis device being adapted to output signal excursions which are created by ionized gas components of the expiration air which hit the detection device,
the analysis device being adapted to determine a portion of the analyte which is to be determined and is contained in the expiration air analyzed by calibration of the signal excursion of the analyte to a signal excursion which is caused by an air moisture of the expiration air.

2. The analysis device according to claim 1, wherein a maximum signal excursion of the analyte is put in relation to a maximum signal excursion of the signal excursion caused by the air moisture of the expiration air, and the portion of the analyte in the expiration air is determined based on a known and constant relative air moisture of expiration air.

3. The analysis device according to claim 1, wherein the analysis device is configured to carry out measurement of a portion of the analyte in the expiration air by calibration of the signal excursion of the analyte to the signal excursion caused by the air moisture of the expiration air with continuously increasing absolute signal excursions and thus immediately after turning on the analysis device.

4. The analysis device according to claim 1, wherein the analyte to be determined is an anesthetic.

5. The analysis device according to claim 4, wherein the analysis device is configured to establish the portion of the anesthetic in the expiration air at predetermined short time intervals, and to indicate measuring values obtained on a display.

6. The analysis device according to claim 1, wherein
the gas components of the expiration air take differently long for a passage through a multi-capillary column and a passage time through the multi-capillary column is referred to as retention time; wherein
the ion mobility spectrometer has an ionization chamber section in which the gas components of the expiration air are ionized and a drift chamber section in which the ionized gas components are accelerated toward the detection device, and a passage time through the drift chamber section is referred to as drift time; and wherein
the analysis device outputs the signal excursions in a chromatogram as a function of the retention time and the drift time.

7. The analysis device according to claim 6, wherein the signal excursion caused by the air moisture of the expiration air is provided in the chromatogram substantially independently of the retention time after a particular drift time and represents a maximum signal excursion in the chromatogram.

8. The analysis device according to claim 6, wherein the signal excursion caused by the air moisture of the expiration air is created by reaction ions formed during ionization of the expiration air and hitting the detection device which excel by a characteristic signal excursion in the chromatogram.

9. The analysis device according to claim 6, further comprising a database in which two values each for the drift time and the retention are stored for different analytes, wherein said values stored in the database define a range in the chromatogram in which the signal excursion for a particular analyte is provided, wherein a drift time axis is scaled to the signal excursion caused by the air moisture of the expiration air.
